(19) United States
(12) Patent Application Publication
(10) Pub. No.: US 2009/0105369 A1
(43) Pub. Date: Apr. 23, 2009

(54) Polymethylmethacrylate Bone Cements

(75) Inventors: SEBASTIAN VOGT, ERFURT (DE); HUBERT BUCHNER, REINHEIM (DE)

Correspondence Address: NORRIS, MCLAUGHLIN & MARCUS, PA, 875 THIRD AVENUE, 18TH FLOOR, NEW YORK, NY, 10022, US

(73) Assignee: HERAEUS MEDICAL GMBH, WEHRHEIM (DE)

(21) Appl. No.: 12/255,397

(22) Filed: Oct. 21, 2008

(30) Foreign Application Priority Data
Oct. 22, 2007 (DE) 10 2007 050 768.4

(51) Int. Cl. A61L 24/04

(52) U.S. Cl. 523/116

(57) ABSTRACT

What is described is a PMMA bone cement, containing at least one homopolymer or one copolymer having a glass transition temperature of more than 45° C., and at least one biocompatible elastomer that has a glass transition temperature of less than 37° C. and is soluble or insoluble in alkylmethacrylates and/or alkyldimethacrylates and/or alkyltrimethacrylates and/or alkyltetramethacrylates and has a residual monomer content of less than 5 percent. The PMMA bone cement is preferably used as self-curing plastic material for the fixation of primary total articular endoprostheses and revision total articular endoprostheses. Moreover, the PMMA bone cement can be used as self-curing filling material for vertebroplasty, kyphoplasty or femoral neck augmentation as well as for the production of temporary place-holders for two-stage revisions of total articular endoprostheses.

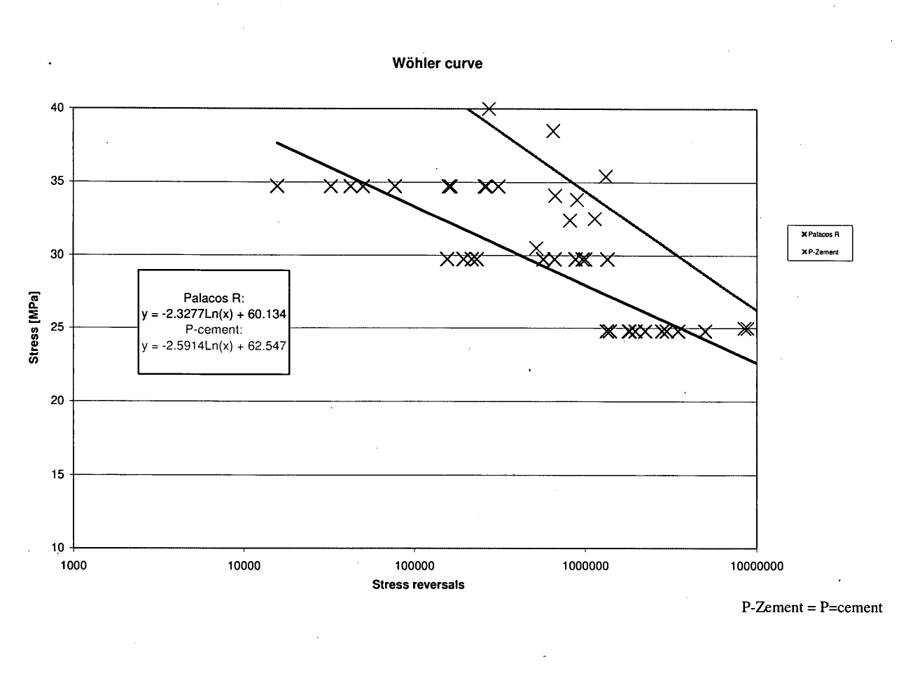

The subject matter of the invention are polymethylmethacrylate bone cements (PMMA bone cements) having increased impact resistance and fatigue life as compared to conventional PMMA bone cements, in particular 2-component PMMA bone cements having powder and liquid component or paste bone cements.

PMMA bone cements have been known for decades and are based on the groundbreaking work of Sir Charnley [J. Charnley, J. Bone Joint Surg. 42 (1960) 28-30].

The basic structure of PMMA bone cements has basically remained unchanged ever since. PMMA bone cements consist of a liquid monomer component and a powder component [G. Lewis, J. Biomed Mater. Res. (Appl. Biomater.) 38 (1997) 155-182]. The monomer component generally contains the monomer, methylmethacrylate, and an activator that is dissolved therein (N,N-dimethyl-p-toluidine). The powder component consists of one or more polymers that are made by polymerisation, preferably suspension polymerisation, based on methylmethacrylate and comonomers, such as styrene, methylacrylate or similar monomers, a radio-opaquer, and the initiator, dibenzoylperoxide. When mixing the powder component with the monomer component, swelling of the polymers of the powder component in the methylmethacrylate leads to the formation of a dough that can be deformed plastically. Simultaneously, the activator, N,N-dimethyl-p-toluidine, reacts with the dibenzoylperoxide which decomposes while forming radicals. The radicals thus formed initiate the radical polymerisation of the methylmethacrylate. Upon advancing polymerisation of the methylmethacrylate, the viscosity of the cement dough increases until the cement dough solidifies and is thus cured.

Two-component paste cements also have become known as alternatives to the powder-liquid system.

The fundamental mechanical requirements for PMMA bone cements, such as 4-point flexural strength, flexural modulus, and compressive strength are described in ISO 5833. Another important mechanical parameter of the PMMA bone cements is the impact resistance. This property characterises the ability of the PMMA bone cements to resist the impact of rapidly-acting mechanical forces. The impact resistance of the PMMA bone cement has particular significance for the expansion of the application field of PMMA bone cements to include kyphoplasty, vertebroplasty, and, in particular, femoral neck augmentation. Moreover, the impact resistance is closely related to the mechanical long fatigue life of the PMMA bone cement. It is therefore desirable to have a PMMA bone cement with improved ability to resist the impact of rapidly-acting mechanical forces and improved fatigue life.

The invention is based on the object to develop a PMMA bone cement that possesses increased impact resistance and increased fatigue life as compared to conventional PMMA bone cements, but simultaneously meets the minimal 4-point flexural strength, flexural modulus, and compressive strength requirements. It is mandatory for the impact resistant PMMA Bone cement to be biocompatible.

The object was met according to the invention by polymethylmethacrylate bone cements (PMMA bone cements) according to claim 1. These are 2-component PMMA bone cements having powder and liquid components or paste bone cements. The bone cements are characterised by at least one homopolymer or one copolymer having a glass transition temperature of at least 45° C. and at least one biocompatible elastomer that has a glass transition temperature of no more than 37° C. and is soluble or capable of swelling in alkylmethacrylates and/or alkyldimethacrylates and/or alkyltrimethacrylates and/or alkyltetramethacrylates and has a residual monomer content of less than 5 percent, being present in the PMMA bone cement. Combining one homopolymer or copolymer having a glass transition temperature of at least 45° with at least one elastomer having a glass transition temperature of no more than 37° C. surprisingly results in the formation of a dimensionally stable PMMA bone cement which, on the one hand, meets the minimal mechanical requirements according to ISO 5833, and, on the other hand, has clearly increased impact resistance. Polymethylmethacrylate is preferred as homopolymer and poly-methylmethacrylate-co-methyl-acrylate and poly-methylmethacrylate-co-styrene are preferred as copolymers. The scope of the invention also includes copolymers made up of alkylmethacrylates other than methylmethacrylate. It is also essential that the elastomer must have a residual monomer content of less than 5 percent such that the PMMA bone cement does not have any toxic effects.

The elastomer is preferably provided in the cement powder in particulate form having a preferred particle size in the range of 5-500 μm. The scope of the invention also allows for the elastomer to be suspended in the particles of the homopolymer or copolymer.

It is useful for the elastomer to be dissolved in the monomer or monomer mixture or to be suspended in the monomer or monomer mixture in the form of swollen particles.

It is also feasible that the elastomer is dissolved in a cement paste or suspended in the cement paste in the form of swollen particles.

Elastomers having a glass transition temperature of less than 0° C. are preferred.

In particular acrylate-caoutchouc, ethylene-acrylate-caoutchouc, ethylene-propylene-terpolymer, ethylene-vinylacetate-copolymer, polybutadiene, polyisoprene, butylcaoutchouc, natural caoutchouc, styrene-butadiene-caoutchouc, and polynorbornene are possible as elastomers.

Moreover, the preferred elastomer content in the polymethylmethacrylate bone cement is 0.1-20.0 mass percent. An elastomer content of 2.0-5.0 mass percent is particularly preferred.

The PMMA bone cement according to the invention is used as self-curing plastic material that is provided for the fixation of primary total articular endoprostheses and revision total articular endoprostheses.

Moreover, the PMMA bone cement according to the invention is used as self-curing filling material that is provided for vertebroplasty, kyphoplasty, and femoral neck augmentation.

The PMMA bone cement can also be used according to the invention for the manufacture of temporary place-holders for two-stage revisions of total articular endoprostheses.

The invention is illustrated in more detail by the examples presented in the following without limiting the scope of the invention. Like in the other parts of the description, specification of parts and percentages refers to the weight unless specified otherwise.

The conventional PMMA bone cement, Palacos® R, was used as reference material.

Firstly, the following cement powder mixtures were made by grinding the components listed in the following in a porcelain ball triturator for 2 hours:

| | Composition of the cement powder | | | |
|---|---|---|---|---|
| | | Poly-methyl- | | |
| | Dibenzoyl- | methacrylate-co- | | Polystyrene-co- |
| Example no. | peroxide | methylacrylate | ZrO₂ | butadiene |
| Palacos ® R | 0.40 g | 33.70 g | 5.90 g | — |
| 1 | 0.39 g | 32.86 g | 5.75 g | 1.00 g |
| 2 | 0.38 g | 32.02 g | 5.60 g | 2.00 g |
| 3 | 0.36 g | 30.33 g | 5.31 g | 4.00 g |

The poly-methyl-methacrylate-co-methylacrylate had a glass transition temperature of 65° C. and the poly-styrene-co-butadiene (BAYMOND) used herein had a glass transition temperature of less than 0° C. The residual monomer content of the poly-styrene-co-butadiene was <3%, as determined by gas chromatography. It was provided in particulate form and its particle size was in the range of 63-250 μm.

Then 40 g cement powder each were mixed with 20 ml methylmethacrylate in which 1.0 mass-% N,N-dimethyl-p-toluidine had been dissolved. This lead to the formation of a dough that was streaked out into square flat hollow moulds (height 3 mm), in which it cured within a few minutes. Strips (75 mm×10 mm×3 mm) were sawed from the cured cement plates. The flexural strength and the impact resistance of the cement strips were determined in accordance with the Dynstat method.

| | Flexural | Impact |
|---|---|---|
| Example no. | strength [MPa] | resistance [J/m²] |
| Palacos ® R | 94.7 ± 1.9 | 4.9 ± 0.4 |
| 1 | 91.6 ± 4.4 | 5.5 ± 1.3 |
| 2 | 91.0 ± 3.1 | 7.8 ± 0.7 |
| 3 | 76.5 ± 3.4 | 8.2 ± 1.3 |

The 4-point flexural strength and the flexural modulus were determined using a Zwick Universal testing apparatus. The compressive strength was determined using cylinder-shaped bodies (h=12 mm, d=6 mm). ISO5833 requires PMMA bone cements to have a minimal strength of ≧50 MPa in the 4-point flexural strength test and a flexural modulus of ≧1800 MPa as well as a minimal compressive strength of ≧70 MPa.

| | | 4-point flexural strength | |
|---|---|---|---|
| | Compressive strength | Flexural strength | Flexural |
| Example no. | [MPa] | [MPa] | modulus [MPa] |
| Palacos ® R | 91.5 ± 3.1 | 65.3 ± 1.9 | 2793 ± 206 |
| 1 | 88.9 ± 2.9 | 59.4 ± 1.1 | 2394 ± 119 |
| 2 | 81.0 ± 3.0 | 54.6 ± 1.5 | 2242 ± 140 |
| 3 | 103.8 ± 3.4* | could not be | 1963 ± 37 |
| | | determined** | |

*Test body was deformed, but not broken
**Test bodies are not broken, test bodies were only deformed

The results presented in the table show that the minimal mechanical requirements according to ISO5833 were met by the PMMA bone cements of examples 1 and 2. The same is to be presumed to be the case for example 3 as well, although no breakage of the test bodies occurred.

Cylinder-shaped test bodies made of Palacos® R and the cements of examples 1 and 2 were tested for their in-vitro cytotoxicity in accordance with ISO10993-5. The test bodies showed no cytotoxic properties under the test conditions used.

Moreover, 2.0 g methacrylate group-terminated poly-styrene-co-butadiene were dissolved in 20 ml methylmethacrylate containing 1.0% N,N-dimethyl-p-toluidine. This monomer solution was mixed with 40.0 g cement powder containing 0.4 g dibenzoylperoxide, 33.7 g poly-methyl-methacrylate-co-methylacrylate, and 5.9 g zirconium dioxide. This lead to the formation of a cement dough that cured after approx. 8 minutes. The impact resistance was comparable to that of example 2.

In addition, the fatigue behaviour of the PMMA bone cement of example 1 was tested. For this purpose, strips (75 mm×10 mm×3 mm) were produced and stored in distilled water for 4 weeks at 37° C. Subsequently, the long fatigue life at three different load levels was determined, whereby the frequency was 5 Hz. The figure below shows the Wöhler curve of the PMMA bone cement of example 1 tested here, termed P-cement herein, compared to the Wöhler curve of Palacos® R.

1. Two-component PMMA bone cement having a powder and a liquid component, or a paste bone cement, comprising
(a) at least one homopolymer or one copolymer having a glass transition temperature of at least 45° C., and
(a) at least one biocompatible elastomer that has a glass transition temperature of no more than 37° C. and is soluble or insoluble in alkylmethacrylates, alkyldimethacrylates, alkyltrimethacrylates, or alkyltetramethacrylates, and has a residual monomer content of no more than 5 percent.

2. The PMMA bone cement according to claim 1, wherein the elastomer is preferably provided in the cement powder in particulate form and having a particle size in the range of 5-500 μm.

3. The PMMA bone cement according to claim 1 wherein the elastomer is dissolved in the monomer or monomer mixture or suspended in the monomer or monomer mixture in the form of swollen particles.

4. The PMMA bone cement according to claim 1 wherein the elastomer is dissolved in a cement paste or suspended in the cement paste in the form of swollen particles.

5. The PMMA bone cement according to claim 1 wherein the elastomer has a glass transition temperature of less than 0° C.

6. The PMMA bone cement according to claim 1 wherein the elastomer is selected from the group consisting of acrylate-caoutchouc, ethylene-acrylate-caoutchouc, ethylene-propylene-terpolymer, ethylene-vinylacetate-copolymer, polybutadiene, polyisoprene, butylcaoutchouc, natural caoutchouc, styrene-butadiene-caoutchouc, and polynorbornene.

7. The PMMA bone cement according to claim 1 wherein the elastomer is present in the polymethylmethacrylate bone cement at 0.1 to 20.0 mass percent.

8. The PMMA bone cement according to claim 7, wherein the elastomer is present in the polymethylmethacrylate bone cement at 2.0 to 5.0 mass percent.

9. (canceled)

10. (canceled)

11. (canceled)
